(19) United States
(12) Patent Application Publication
(10) Pub. No.: US 2019/0180883 A1
(43) Pub. Date: Jun. 13, 2019

(54) MILESTONE DETECTION SENSING

(71) Applicant: TeleTracking Technologies, Inc., Pittsburgh, PA (US)

(72) Inventor: Joseph Christopher Schuck, McMurray, PA (US)

(21) Appl. No.: 15/837,525

(22) Filed: Dec. 11, 2017

(51) Int. Cl. G16H 80/00

(52) U.S. Cl. CPC G16H 80/00

(57) ABSTRACT

One embodiment provides a method, including: receiving, using at least one sensor, user input, wherein the user input comprises at least one of audio input and visual input; determining, using a processor, whether at least a portion of the received user input corresponds to a milestone event; and updating, responsive to determining that the at least a portion of the received user input corresponds to a milestone event, a system with the milestone event. Other aspects are described and claimed.

### BACKGROUND

In many workplace environments (e.g., hospitals, ambulances, other businesses, etc.), activities and events occur that need to be monitored and logged. For example, in a medical setting such as a hospital operating room, a patient's status and condition during a surgical procedure needs to be tracked (e.g., when has a patient received anesthesia, when has the procedure begun/concluded, etc.) and provided into a corresponding database (e.g., the patient's file in the hospital's system, a status update database, etc.). Users frequently utilize information handling devices (“devices”), for example smart phones, tablet devices, laptop and personal computers, and the like, to manually provide these informational updates to the system.

### BRIEF SUMMARY

In summary, one aspect provides a method comprising: receiving, using at least one sensor, user input, wherein the user input comprises at least one of audio input and visual input; determining, using a processor, whether at least a portion of the received user input corresponds to a milestone event; and updating, responsive to determining that the at least a portion of the received user input corresponds to a milestone event, a system with the milestone event.

Another aspect provides an information handling device, comprising: a processor; at least one sensor operatively coupled to the processor; a memory device that stores instructions executable by the processor to: receive user input, wherein the user input comprises at least one of audio input and visual input; determine whether at least a portion of the received user input corresponds to a milestone event; and update, responsive to determining that the at least a portion of the received user input corresponds to a milestone event, a system with the milestone event.

A further aspect provides a product, comprising: a storage device that stores code, the code being executable by a processor and comprising: code that receives user input, wherein the user input comprises at least one of audio input and visual input; code that determines whether at least a portion of the received user input corresponds to a milestone event; and code that updates, responsive to determining that the at least a portion of the received user input corresponds to a milestone event, a system with the milestone event.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example method of updating a system.

FIG. 2 illustrates an example positional arrangement of a multi-modal sensor according to an embodiment.

FIG. 3 illustrates an example of device circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

Many procedures have different milestones or steps that are completed when performing the procedure, particularly in a hospital environment (e.g., emergency room, doctor's office, long-term care, in-patient services, operating room, etc.). For example, a surgical procedure may have steps related to anesthesia, incisions, stitches, and the like. As another example, a doctor may complete rounds which include visitations to different patients and rooms. Thus, the steps may include entering a room with a patient, spending time with the patient, exiting the room, and the like. Many hospital environments use the information for completion of particular steps to update different systems. For example, a hospital may provide a patient status display in a waiting-room identifying the status of the procedure. As another example, a scheduling database may use the information regarding completion of steps to assist in scheduling doctors, nurses, or other staff.

In current systems, identification of the completeness of the different steps in the procedure may require the manual provision of updates to a system, for example, the doctor, nurse, or other staff member, may have to provide input to a system indicating that a particular step has been completed. Manual provision of event updates to a system may be cumbersome and inconvenient in a variety of situations. For example, in an operating room setting, a surgeon cannot easily pause an active procedure and provide updates to a system when a major event, or “milestone”, occurs (e.g., when a surgeon has begun the surgery by making an incision, when a patient's vitals begin to fall, etc.). Thus, the hospital usually provides an extra staff member that provides the updates to the system, which can be costly.

Additionally, timestamp data associated with the event may be imprecise if, for example, a user did not immediately update the system as the event occurred (e.g., distracted by the procedure, forgot to update the system, because a user had to complete a necessary task prior to inputting the information such as removing their gloves or completing an important note, etc.). Additionally, in the event of a complication of the procedure, it may not be feasible, expected, or safe, for a staff member to update the system, rather, the staff member should be focused on addressing the complication.

Some conventional system provide radio-frequency identification (RFID) tags that may be provided or attached to objects and/or individuals to track not only their whereabouts but also when the object or individual arrived at a particular location. For example, when a surgeon equipped with an RFID tag enters an operating room, a system may be notified that the surgeon has arrived in the operating room at a particular time by identifying the location of the RFID tag and when the RFID tag entered that location. However, although RFID tagging may be able to identify a location of an object or a user, this tagging method is unable to provide information regarding the completion of events in a user's task. For example, although RFID tagging may be able to identify that a surgeon is in the operating room, this method will be unable to identify that a surgeon has begun surgery.

Accordingly, an embodiment provides a method for automatically updating a system when a milestone event has been detected. In an embodiment, user input (e.g., audio input, visual input, a combination thereof, etc.) may be received by at least one sensor (e.g., a multi-modal sensor, etc.) located in the vicinity of the user (e.g., a sensor may be positioned in the room where an activity occurs, etc.). An embodiment may then determine whether the input corresponds to an indication of a milestone event and, responsive to making a positive determination, an embodiment may update a system with the milestone event (e.g., log the milestone event in a database, provide a visual indication that the milestone event was logged, contact another individual and inform them of the milestone event, etc.). Such a method may eliminate the need for users to manually provide updates to a system when a milestone event occurs. Additionally, such a method may be able to update a system in substantially real-time as the event occurs, which consequently makes any timestamp data associated with the event more accurate.

The illustrated example embodiments will be best understood by reference to the figures. The following description is intended only by way of example, and simply illustrates certain example embodiments.

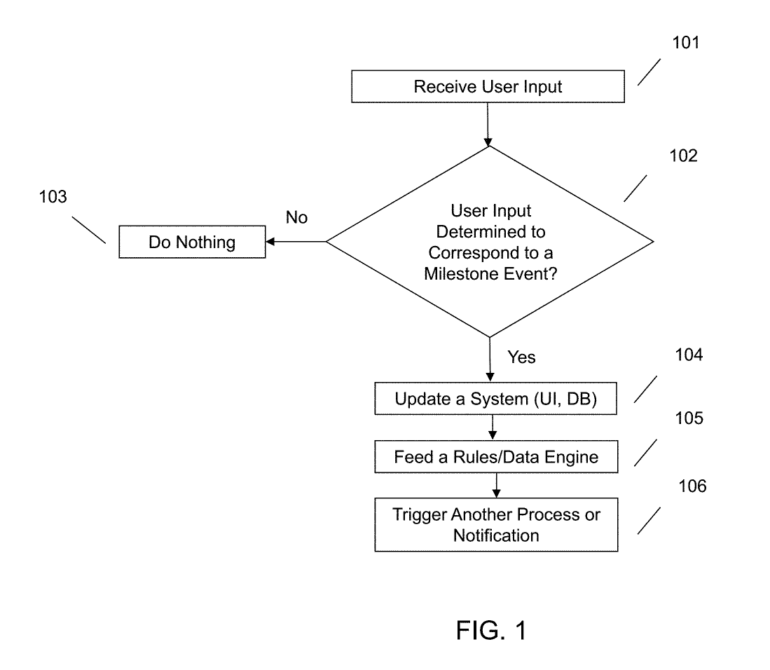

Referring now to FIG. 1, an embodiment may update a system responsive to determining that user input corresponding to a milestone event has been received. At 101, an embodiment may receive user input at a sensor. In an embodiment, the user input may be audible input, gesture input (e.g., a predefined gesture, a generic movement pattern, etc.), a combination thereof, and the like. For example, the user may provide a voice input that a milestone event has occurred. As another example, the user may provide a gesture that indicates that a milestone event has occurred. The user input may cause an embodiment to perform a function in a system, as explained in more detail below.

In an embodiment, the sensor may be a single-modal sensor (e.g., a sensor capable of detecting a singular input type such as only audible input or only visual input, etc.) or a multi-modal sensor (e.g., a sensor capable of detecting and recognizing a plurality of input types such as audible input, visual input, gesture input, etc.). The multi-modal sensor may also be capable of additional functionality such as infrared image capture, facial recognition, illumination, and the like. In an embodiment, a plurality of single or multi-modal sensors may be utilized (e.g., multiple sensors may be positioned around a room and may relay any received input to a system, etc.). In an embodiment, the sensor may be configured to continuously receive user input by maintaining the sensor in an active state. The sensor may, for example, continuously detect voice input data even when other sensory functions (e.g., light sensors, other microphones, etc.) are inactive. Alternatively, the sensor may remain in an active state for a predetermined amount of time (e.g., 30 minutes, 1 hour, 2 hours, etc.), or the sensor may “wake-up” in response to a trigger word or receipt of user input.

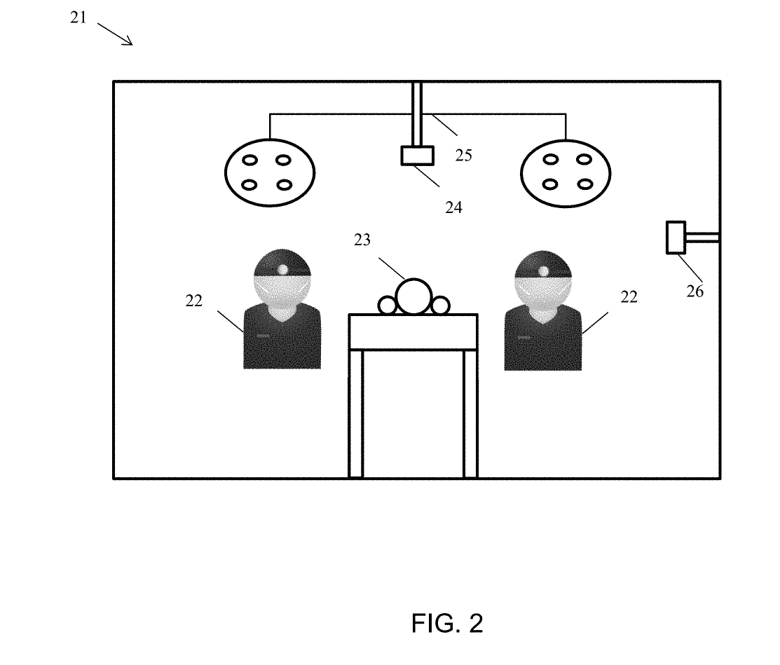

In an embodiment, the one or more sensors may be positioned at a proximate location to a user. In the context of this application, a proximate location may refer to any location around a user in which the sensor is capable of properly receiving user inputs. As a non-limiting example, referring now to FIG. 2, an operating room setting 21 is illustrated in which medical personnel 22 (e.g., surgeons, etc.) are positioned around an anesthetized patient 23 laying prone on a table. In the illustrated example, a sensor 24 is positioned above the patient and is operatively coupled to a lighting complex 25 comprising two adjustable lighting units. Such a position may enable the sensor 24 to not only adequately receive voice inputs but also enable the sensor to appropriately identify visual or gesture inputs provided by the medical personnel 22 or other staff (not pictured) in the vicinity. Additionally or alternatively, in another embodiment, additional sensors 26 may be positioned at other locations in in the room (e.g., the wall of the room, etc.).

At 102, an embodiment may determine whether at least a portion of the received user input corresponds to a milestone event. In the context of this application, a milestone event may be any event that has been identified to be of some significance. For example, a milestone event may refer to the arrival of a patient in a room, the beginning of a surgical procedure, and the like. An identification of milestone events may be programmed into the system. For example, a hospital administrator may identify events that should be considered milestone events. Milestone events may also be learned by the system. For example, if a milestone event for an operating room is identified as the patient entering the room, the system may infer that a doctor entering the room should also be identified as a milestone event even though not directly identified as a milestone event to the system. Additionally, identified milestone events may be extrapolated to other settings. For example, if milestone events are previously identified for an operating room, the system may extrapolate those events to correspond to events that may occur in patient rooms.

In an embodiment, the determining step may be performed by identifying whether the user input comprises an activation cue. The activation cue may be a triggering action that informs the system that a particular milestone event is about to occur or has just occurred. In an embodiment, the activation cue may be, for example, a trigger word, a trigger gesture, a trigger movement, and the like. In an embodiment, the activation cues may be stored in one or more accessible storage databases (e.g., remote storage, local storage, cloud storage, etc.) that may contain associations between the activation cues and known milestone events. For example, a thumbs up trigger gesture may be associated with the initiation of a procedure; a trigger word such as “end procedure” may be associated with the completion of a procedure, etc. A single gesture, activation word, or other activation cue may be used by the system to identify that the input is directed at the system. For example, a user may provide the same activation word or phrase which triggers the system to “listen” for the milestone update, whenever a milestone update is to occur.

In an embodiment, the identification of an activation cue in a user input may be achieved by comparing the user input to the one or more databases of associations. For example, if an embodiment received the audible input “alright, everyone is here, let's go ahead and begin the procedure”, an embodiment may be able to compare the user input against the one or more databases to determine if at least a portion of the user input corresponds to a known activation cue. In this situation, an embodiment may recognize that the portion of the input stating “begin the procedure” is above a predetermined threshold of similarity to the stored trigger word activation cue “begin procedure” that corresponds to the milestone event of procedure initiation. Details regarding the different activation cues are provided below.

In an embodiment, an activation cue may be associated with a trigger word. The trigger word may include one or more words, for example, the trigger word may include a phrase or multiple words rather than a single word. The trigger word may include a description of an activity being performed or may include a statement that an event that has occurred. For example, the trigger word may be a description of an event such as “starting procedure” or identification of an event such as “patient is recovering”. In one embodiment, the trigger word may be a preset or default word or may be programmed by a user. For example, a user may choose a particular word or phrase to be used to cause an embodiment to perform the function on the system. Different users may select or program different trigger words. For example, in a hospital setting, one surgeon may prefer a trigger word such as “beginning procedure” while another surgeon prefers the trigger word “starting procedure.” Different trigger words for different users may reflect personal preferences of a user and/or help a device to differentiate between users. For example, using the aforementioned example, an embodiment may identify which surgeon performed the operation and provided the input to the system based upon the trigger words used.

In an embodiment, an activation cue may be associated with a trigger gesture. The trigger gesture may be a predefined static gesture (e.g., a thumbs up, etc.) or a predefined dynamic, moving gesture (e.g., a wave of a hand, etc.). In an embodiment, each trigger gesture may correspond to a particular milestone event description or identification. For example, a thumbs up may indicate that a procedure has started whereas a thumbs down may indicate that something has gone wrong. In one embodiment, the trigger gesture may be a preset or default gesture or may be programmed by a user. For example, a user may choose a particular gesture or movement pattern to be used to cause an embodiment to perform the function on the system. Different users may select or program different trigger gestures. For example, in a hospital setting, one surgeon may prefer a trigger gesture such as thumbs up to indicate that a procedure has begun whereas another surgeon may prefer the thumbs up gesture to indicate that a procedure was successful. Different gestures for different users may reflect personal preferences of a user and/or help a device to differentiate between users. For example, using the aforementioned example, an embodiment may identify which surgeon performed the operation and provided the input to the system based upon the trigger gestures used.

In another embodiment, the activation cue may be associated with a trigger movement or trigger word. The trigger movement may simply be an action committed by a user during the natural course of completing a task that a system has been trained to recognize as being a milestone event. For example, in an operating room setting, a system may continuously monitor a surgeon's movements and determine that when a surgeon has picked up a scalpel, or has made an incision into a patient, that a procedure has begun. The surgeon in this example need not have provided any other additional trigger word or gesture. Similarly, the trigger word may simply be a word or phrase spoken by the user during the natural course of completing a task. For example, when the surgeon is about to start an incision, the surgeon may say “scalpel” to another staff member in the room. The system may identify this word as an indication that the incision is about to begin. In other words, the system may not require that the users provide unique words, phrases, or gestures to specifically identify to the system that a milestone has been completed. Rather, the system may continuously monitor the user's movements and voice input and infer which milestones have been completed based upon gestures and voice input that is received during the natural course of procedure completion.

In an embodiment, one or more activation cues may be received and considered when determining whether at least a portion of the user input corresponds to a milestone event. For example, an embodiment may require the receipt of two or more activation cues corresponding to the same milestone event to update the system. For example, an embodiment may initially receive the trigger word “starting surgery now” but may not take an additional action (e.g., update the system with the milestone event, etc.) until another activation cue associated with surgery initiation is received. If an embodiment receives another activation cue corresponding to surgery imitation (e.g., such as the trigger movement of a surgeon making an incision into a patient with a scalpel, etc.) an embodiment may then positively identify that the user input corresponds to the milestone event of surgery initiation.

The determination that a milestone event may occur, is occurring, or just occurred may also be based upon other information that can be detected and/or received by the system. For example, if the users and objects have the RFID tags, as discussed above, the system may take into account the location of objects with respect to other objects. For example, the surgeon entering the room may be based in part on the detection of the RFID tag associated with the surgeon within the room. As another example, a determination of a procedure starting may be based in part on the detection of surgical implements being in proximity to the surgeon. As a final example, a determination of a procedure starting may be based in part of the detection of a doctor being in proximity to a patient or patient bed.

Responsive to determining, at 102, that at least a portion of the user input corresponds to a milestone event, an embodiment may automatically update, at 104, a system with the milestone event. In the context of this application, the system may be virtually any system that comprises files able to be updated (e.g., a patient tracking system comprising a plurality of patient logs, a scheduling system, a bed availability system, etc.). In the context of this application, automatically updating the system may refer to an update provided to the system without any additional user input. Responsive to determining that at least a portion of the user input does not correspond to a milestone event, an embodiment may, at 103, take no action (e.g., not update the system, etc.).

In an embodiment, an update provided to the system may refer to a logging of the milestone event. For example, if the milestone event corresponding to surgery initiation has been determined, an embodiment may update a patient's log to indicate that they have begun surgery. In an embodiment, the system may be updated in substantially real-time as the milestone event is determined. For example, upon determining that a patient has begun surgery, an embodiment may immediately update the system with that information. In an embodiment, the updating may comprise recording, in the system, additional aspects associated with the milestone event (e.g., a timestamp of when the milestone event was determined, an identification of who provided the activation cue corresponding to the milestone event, etc.). The additional aspects recorded may be fed, at 105, into a rules and/or data engine capable of storing and/or performing additional tasks using the additional aspects.

In an embodiment, at 106, an audio or visual indication associated with the update may be provided to one or more devices having access to the system. For example, regarding a visual indication, an embodiment may provide, on a graphical user interface, an animation indicative of the update (e.g., a check to a box in a patient's log corresponding to the milestone event, an icon status change corresponding to the patient, etc.), a textual message describing the update, and/or the like. In another embodiment, regarding an audible notification, an embodiment may provide a sound indicative of an update action (e.g., a preselected “ding”, etc.), an audible description of the update, and/or the like. In an embodiment, information associated with the update may be viewable to other devices with access to the system. For example, a nurse or administrator having access to the system may access a patient's log from a mobile device (e.g., smart phone, tablet, etc.) and be notified of the update to the patient's log. In an embodiment, the audio or visual notification may be provided to the other devices (e.g., a connected tablet may receive a textual message describing the update, etc.). Additionally, updating one system may cause an update to a second system. For example, a system associated with a patient status may be updated, which may then cause another system, for example, a patient status display, to be updated.

The various embodiments described herein thus represent a technical improvement to current system updating techniques. The systems and methods as described herein enable milestone events associated with an activity to be tracked and a system responsible for keeping track of the milestone events to be updated responsive to identifying the occurrence of a milestone event. In an embodiment, a system may receive user input and thereafter determine whether at least a portion of the user input corresponds to a milestone event. Responsive to determining that at least a portion of the user input corresponds to a milestone event, an embodiment may automatically update a system accordingly. Such techniques prevent users from having to manually provide updates to a system for a milestone event.

While various other circuits, circuitry or components may be utilized in information handling devices, with a computer, server, client device or the like, an example device that may be used in implementing one or more embodiments includes a computing device in the form of a computer 300. This example device may be a server used in one of the systems in a hospital network, or one of the remote computers connected to the hospital network. Components of computer 300 may include, but are not limited to, a processing unit 320, a system memory 330, and a system bus 322 that couples various system components including the system memory 330 to the processing unit 320. Computer 300 may include or have access to a variety of computer readable media, including databases. The system memory 330 may include non-signal computer readable storage media, for example in the form of volatile and/or nonvolatile memory such as read only memory (ROM) and/or random access memory (RAM). By way of example, and not limitation, system memory 330 may also include an operating system, application programs, other program modules, and program data.

A user can interface with (for example, enter commands and information) the computer 300 through input devices 350. A monitor or other type of device can also be connected to the system bus 322 via an interface, such as an output interface 360. The computer may include a database 340, e.g., if it is part of the warehouse layer in FIG. 1. In addition to a monitor, computers may also include other peripheral output devices. The computer 300 may operate in a networked or distributed environment using logical connections to one or more other remote device(s) 380 such as other computers. The logical connections may include network interface(s) 370 to a network, such as a local area network (LAN), a wide area network (WAN), and/or a global computer network, but may also include other networks/buses.

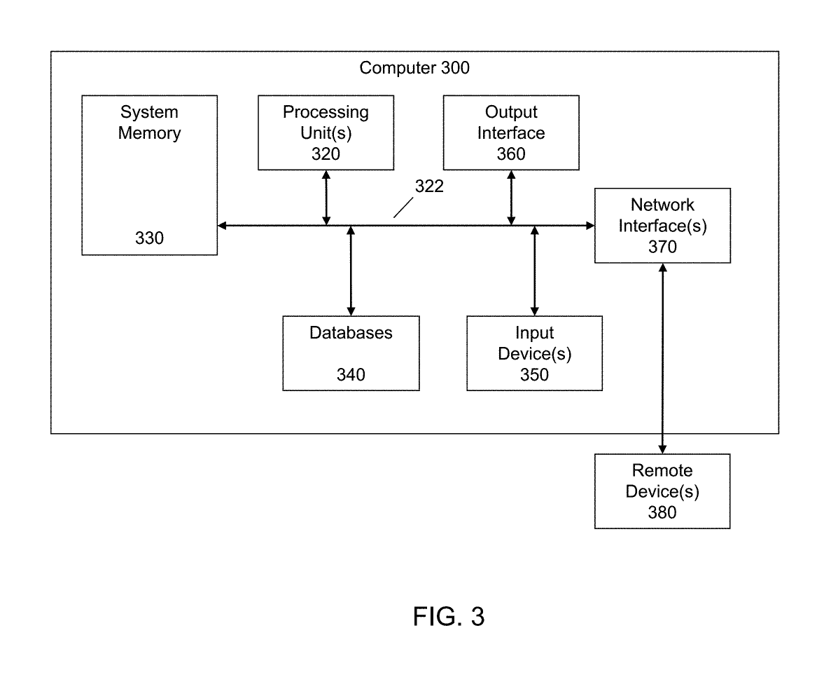

Information handling device circuitry, as for example outlined in FIG. 3, may be used in client devices such as a personal desktop computer, a laptop computer, or smaller devices such as a tablet or a smart phone. In the latter cases, i.e., for a tablet computer and a smart phone, the circuitry outlined in FIG. 3 may be adapted to a system on chip type circuitry. The device, irrespective of the circuitry provided, may provide and receive data to/from another device, e.g., a server or system that coordinates with various other systems. As will be appreciated by one having ordinary skill in the art, other circuitry or additional circuitry from that outlined in the example of FIG. 3 may be employed in various electronic devices that are used in whole or in part to implement the systems, methods and products of the various embodiments described herein.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and “non-transitory” includes all media except signal media.

Program code embodied on a storage medium may be transmitted using any appropriate medium, including but not limited to wireless, wireline, optical fiber cable, RF, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method comprising:
receiving, using at least one sensor, user input, wherein the user input comprises at least one of audio input and visual input;
determining, using a processor, whether at least a portion of the received user input corresponds to a milestone event; and
updating, responsive to determining that the at least a portion of the received user input corresponds to a milestone event, a system with the milestone event.

2. The method of claim 1, wherein the receiving comprises continuously receiving the user input.

3. The method of claim 1, wherein the determining comprises identifying, from the user input, an activation cue.

4. The method of claim 3, wherein the activation cue is at least one triggering action selected from the group consisting of a trigger word, a trigger gesture, and a trigger movement.

5. The method of claim 3, wherein the identifying comprises comparing the user input to a database of stored activation cues and identifying that at least a portion of the received user input has a predetermined threshold level of similarity with at least one stored activation cue.

6. The method of claim 5, wherein the activation cue comprises at least two activation cues and wherein the determining comprises determining that the at least two activation cues correspond to the same milestone event.

7. The method of claim 1, wherein the updating comprises providing at least one of a visual indication and an audible notification that the system was updated.

8. The method of claim 1, wherein the updating comprises recording, in the system, at least one of a time associated with the milestone event and a user that provided the user input.

9. The method of claim 1, wherein the updating comprises notifying at least one other user of the milestone event.

10. The method of claim 1, wherein the at least one sensor comprises at least one multi-modal sensory device.

11. An information handling device, comprising:
a processor;
at least one sensor operatively coupled to the processor;
a memory device that stores instructions executable by the processor to:
receive user input, wherein the user input comprises at least one of audio input and visual input;
determine whether at least a portion of the received user input corresponds to a milestone event; and
update, responsive to determining that the at least a portion of the received user input corresponds to a milestone event, a system with the milestone event.

12. The information handling device of claim 11, wherein the instructions executable by the processor to receive comprise instructions executable by the processor to continuously receive the user input.

13. The information handling device of claim 11, wherein the instructions executable by the processor to determine comprise instructions executable by the processor to identify, from the user input, an activation cue.

14. The information handling device of claim 13, wherein the activation cue is at least one triggering action selected from the group consisting of a trigger word, a trigger gesture, and a triggering movement.

15. The information handling device of claim 13, wherein the instructions executable by the processor to identify comprise instructions executable by the processor to compare the user input to a database of stored activation cues and identify that at least a portion of the received user input has a predetermined threshold level of similarity with at least one stored activation cue.

16. The information handling device of claim 15, wherein the activation comprises at least two activation cues and wherein the instructions executable by the processor to determine comprise instructions executable by the processor to determine that the at least two activation cues correspond to the same milestone event.

17. The information handling device of claim 11, wherein the instructions executable by the processor to update comprise instructions executable by the processor to provide at least one of a visual indication and an audible notification that the system was updated.

18. The information handling device of claim 11, wherein the instructions executable by the processor to update comprise instructions executable by the processor to record, in the system, at least one of a time associated with the milestone event and a user that provided the user input.

19. The information handling device of claim 11, wherein the instructions executable by the processor to update comprise instructions executable by the processor to notify at least one other user of the milestone event.

20. A product, comprising:
a storage device that stores code, the code being executable by a processor and comprising:
code that receives user input, wherein the user input comprises at least one of audio input and visual input;
code that determines whether at least a portion of the received user input corresponds to a milestone event; and
code that updates, responsive to determining that the at least a portion of the received user input corresponds to a milestone event, a system with the milestone event.
